(19) United States
(12) Patent Application Publication
(10) Pub. No.: US 2023/0177443 A1
(43) Pub. Date: Jun. 8, 2023

(54) SYSTEMS AND METHODS FOR AUTOMATED MODELING OF PROCESSES

(71) Applicant: THE GOVERNING COUNCIL OF THE UNIVERSITY OF TORONTO, Toronto (CA)

(72) Inventors: Arik SENDEROVICH, Concord (CA); Opher BARON, Thornhill (CA); Dmitry KRASS, Toronto (CA)

(21) Appl. No.: 18/050,693

(22) Filed: Oct. 28, 2022

Related U.S. Application Data
(60) Provisional application No. 63/263,190, filed on Oct. 28, 2021.

(51) Int. Cl. G06Q 10/067

(52) U.S. Cl. CPC G06Q 10/067

(57) ABSTRACT

Methods and systems for training a simulation model of a process are described. They can include receiving historical event data from a work system, processing the data to estimate system occupancy information and enriching the historical event data, building the model by extracting activities and estimating pathways and routing probabilities, and enhancing the model by removing some of the pathways and estimating durations of activities in the pathways. The simulation model can be used to evaluate the impact of process interventions by either extracting an impact of the interventions from the data if it contains events that correspond to the interventions, or simulating the process with the interventions using the model otherwise. The simulation model can furthermore be used to derive prescriptions by simulating the process with possible interventions and determining which interventions optimize a ratio of a performance function and a cost function.

### CROSS-REFERENCE TO RELATED APPLICATION

This application claims the benefit of and priority to U.S. Provisional Patent Application No. 63/263,190, filed Oct. 28, 2021, entitled SYSTEMS AND METHODS FOR AUTOMATED MODELING OF SERVICE PROCESSES, the entirety of which is hereby incorporated by reference.

### TECHNICAL FIELD

The present disclosure generally relates to analyzing processes, and more specifically to methods and systems for automated modeling of processes to allow making descriptive, comparative, predictive and prescriptive analytics thereof.

### BACKGROUND

Analytical and simulation models have been used extensively to analyze work systems such as service systems. Given that traditional analytical techniques usually only work for relatively simple systems, simulation modeling is often the only feasible solution for real-life processes. However, there are many challenges with simulation models that are often seen as fatal in modern fast-changing work systems.

One challenge is missing log data, which is a very common occurrence. The fact that missing data affects model validation means insights produced by the model cannot be trusted.

Another challenge relates to long development times. Due to the complexity of the systems, it generally takes a long time to produce a useable model. Such developments times are often measured on the order of months for large processes. As such, the process being modeled will likely have changed by the time the model is complete.

A further challenge relates to non-unique representation. Since simulation models are constructed manually, two expert analysts may create very different models for the same system. This reduces trust in the model and causes ambiguity as to which of the models is correct, especially when more than a single performance measure is being used.

There is therefore much room for improvement.

### SUMMARY

Systems and methods are described for creating accurate work system representations automatically via data-driven methods. The mapping from data to the model can be well defined and transparent. The resulting models can be generative in nature, i.e., they can generate new data that resembles the actual data coming from the system under different interventions. Thus, simulations, automatically created based on the original data, can serve as a test bed to study the impact of different interventions, enabling comparative analytics. The simulation can be developed, verified, and validated, using machine learning models that guide the internal sampling process. By using approximations from queueing theory, the system's behaviour can be estimated with adequate levels of accuracy even when data is missing or inaccurate.

According to an aspect, a method for automatically generating a model of a service process is provided. The method includes: receiving historical event data corresponding to activities processed by a service system, said historical event data comprising a plurality of events and, for each event, values corresponding to a plurality of attributes, said plurality of attributes including at least a participant identifier, an activity identifier and a timestamp; processing the historical event data to extract system occupancy information and enhancing the historical event data by adding the system occupancy information as at least one additional context attribute; separating the enhanced historical event data into a training dataset and a testing dataset; building a queueing network model by extracting activities and estimating pathways and routing probabilities from the training dataset; and enhancing the queueing network model by processing the training dataset to group or remove at least some of the pathways and estimate durations of activities in said pathways.

According to an aspect, a system for automatically generating a model of a service process is provided. The system includes: an input module configured to receive historical event data corresponding to activities processed by a service system, said historical event data comprising a plurality of event and, for each event, values corresponding to a plurality of attributes, said plurality of attributes including at least a participant identifier, an activity identifier and a timestamp; a data enhancement module configured to process the historical event data to extract system occupancy information and enhance the historical event data by adding the system occupancy information as at least one additional context attribute; and a model learning module configured to separate the enhanced historical event data into a training dataset and a testing dataset, the model learning module comprising a process discovery submodule configured to build a queueing network model by extracting activities and estimating pathways and routing probabilities from the training dataset, and a queue mining submodule configured to enhance the queueing network model by processing the training dataset to group or remove at least some of the pathways and estimate durations of activities in said pathways.

According to an aspect, a non-transitory computer-readable medium is provided. The computer-readable medium has instructions stored thereon which, when executed by a processor of a computing system, cause the computing system to: receive historical event data corresponding to activities processed by a service system, said historical event data comprising a plurality of events and, for each event, values corresponding to a plurality of attributes, said plurality of attributes including at least a participant identifier, an activity identifier and a timestamp; process the historical event data to extract system occupancy information and enhance the historical event data by adding the system occupancy information as at least one additional context attribute; separate the enhanced historical event data into a training dataset and a testing dataset; build a queueing network model by extracting activities and estimating pathways and routing probabilities from the training dataset; and enhance the queueing network model by processing the training dataset to group or remove at least some of the pathways and estimate durations of activities in said pathways.

According to an aspect, a method for training a simulation model of a process is provided. The method includes: receiving historical event data corresponding to activities processed by a work system, said historical event data comprising a plurality of events and, for each event, values corresponding to a plurality of attributes, said plurality of attributes including at least a participant identifier, an activity identifier and a timestamp; processing the historical event data to estimate system occupancy information and enhancing the historical event data by adding the estimated system occupancy information as at least one additional context attribute; separating the enhanced historical event data into a training dataset and a testing dataset; building the simulation model by extracting activities and estimating participant pathways and routing probabilities between the activities, from the training dataset; and enhancing the simulation model by processing the training dataset to remove at least some of the participant pathways based on frequency, and to estimate durations of activities in the participant pathways.

According to an aspect, a system for training a simulation model of a process is provided. The system includes: an input module configured to receive historical event data corresponding to activities processed by a work system, said historical event data comprising a plurality of event and, for each event, values corresponding to a plurality of attributes, said plurality of attributes including at least a participant identifier, an activity identifier and a timestamp; a data enhancement module configured to process the historical event data to estimate system occupancy information and enhance the historical event data by adding the estimated system occupancy information as at least one additional context attribute; and a model learning module configured to separate the enhanced historical event data into a training dataset and a testing dataset, the model learning module comprising a process discovery submodule configured to build the simulation model by extracting activities and estimating participant pathways and routing probabilities between the activities from the training dataset, and a queue mining submodule configured to enhance the simulation model by processing the training dataset remove at least some of the participant pathways based on frequency, and to estimate durations of activities in the participant pathways.

According to an aspect, a non-transitory computer-readable medium is provided. The non-transitory computer-readable medium has instructions stored thereon which, when executed by a processor of a computing system, cause the computing system to: receive historical event data corresponding to activities processed by a work system, said historical event data comprising a plurality of events and, for each event, values corresponding to a plurality of attributes, said plurality of attributes including at least a participant identifier, an activity identifier and a timestamp; process the historical event data to estimate system occupancy information and enhance the historical event data by adding the estimated system occupancy information as at least one additional context attribute; separate the enhanced historical event data into a training dataset and a testing dataset; build a simulation model by extracting activities and estimating participant pathways and routing probabilities between the activities, from the training dataset; and enhance the simulation model by processing the training dataset to remove at least some of the participant pathways based on frequency, and to estimate durations of activities in said participant pathways.

### BRIEF DESCRIPTION OF THE DRAWINGS

FIG. 1 is a block diagram illustrating a system for automatically modeling a process, according to an embodiment.

FIG. 2 is a schematic illustrating exemplary event data that can be provided to the system of FIG. 1.

FIG. 3 is a table illustrating exemplary event data contained within an event log of an emergency department service system.

FIG. 4 is a flowchart illustrating a method for automatically modeling a process that can be carried out by the system of FIG. 1, according to an embodiment.

FIG. 5 is a flowchart illustrating a method for predicting impact of interventions on a process, according to an embodiment.

FIG. 6 is a flowchart illustrating a method for optimizing intervention in a process, according to an embodiment.

FIGS. 7A and 7B are directed graphs corresponding to exemplary pre-models that can be produced during performance of the method illustrated in FIG. 4.

### DETAILED DESCRIPTION

In the foregoing description, systems and methods will be described for automatically modeling and analyzing processes such as service and manufacturing processes, among others. Processes can generally be modeled as queueing networks which comprise a plurality of interconnected queueing nodes or stations. In such models, jobs or customers arrive at a queue, are processed at the queue (i.e. are provided a service via a resource), and then depart from the queue. Depending on the nature and capacity of the queue, the job or customer may need to wait when arriving at a queue before it is their turn to be processed, and/or it may take some time to process the job or customer once it is their turn.

As can be appreciated, a process can correspond to any process implemented by a work system, such as a service system that provides one or more services using available resources. A work system can be of different types, such as manufacturing systems that assemble finished products from components at one or more stations, supply chain systems that transform and assemble materials into intermediate goods then finished products at one or more facilities. As such, system elements corresponding to jobs/customers, resources, stations/services, etc. can vary depending on the nature of the system. Accordingly, in the following description, terms such as “job”, “customer”, “resource”, “service”, “station”, etc. are intended to describe generic elements of queueing networks, and are not intended to refer to a type of work system in particular. Moreover, similar terms such as “job”/“customer, or “service”/“station”/“node” will be used interchangeably.

In the following description, reference will be made to “events” occurring within a process. It should be appreciated that an event can correspond to any transaction within a queueing network that relates to how a customer travels through the queueing network and/or how the customer is processed at any given node. An event can, for example, correspond to a customer or a component arriving at a node, a customer or a component being processed at a node, and/or a customer or a component leaving a node, among other possibilities.

One or more systems described herein may be implemented in computer program(s) executed on processing device(s), each comprising at least one processor, a data storage system (including volatile and/or non-volatile memory and/or storage elements), and optionally at least one input and/or output device. “Processing devices” encompass computers, servers and/or specialized electronic devices which receive, process and/or transmit data. As an example, “processing devices” can include processing means, such as microcontrollers, microprocessors, and/or CPUs, or be implemented on FPGAs. For example, and without limitation, a processing device may be a programmable logic unit, a mainframe computer, a server, a personal computer, a cloud-based program or system, a laptop, a personal data assistant, a cellular telephone, a smartphone, a wearable device, a tablet, a video game console, or a portable video game device.

Each computer program can be implemented in a high-level programming and/or scripting language, for instance an imperative e.g., procedural, or object-oriented, or a declarative e.g., functional or logic, language, to communicate with a computer system. However, a program can be implemented in assembly or machine language if desired. In any case, the language may be a compiled or an interpreted language. Each such computer program can be stored on a storage media or a device readable by a general or special purpose programmable computer for configuring and operating the computer when the storage media or device is read by the computer to perform the procedures described herein. In some embodiments, the system can be embedded within an operating system running on the programmable computer.

Furthermore, the system, processes and methods of the described embodiments are capable of being embodied in a computer program product comprising a computer-readable medium having computer-usable instructions which, when executed, cause one or more processors to implement the system and/or carry out steps of the processes and methods. The computer-usable instructions can be in various forms including compiled and non-compiled code.

The processor(s) can be used in combination with storage medium, also referred to as “memory” or “storage means”. Storage medium can store instructions, algorithms, rules and/or trading data to be processed. Storage medium encompasses volatile or non-volatile/persistent memory, such as registers, cache, RAM, flash memory, ROM, diskettes, compact disks, tapes, chips, as examples only. The type of memory can be chosen according to the desired use, whether it should retain instructions, or temporarily store, retain or update data. Steps of the proposed method can be implemented as software instructions and algorithms, stored in computer memory and executed by processors.

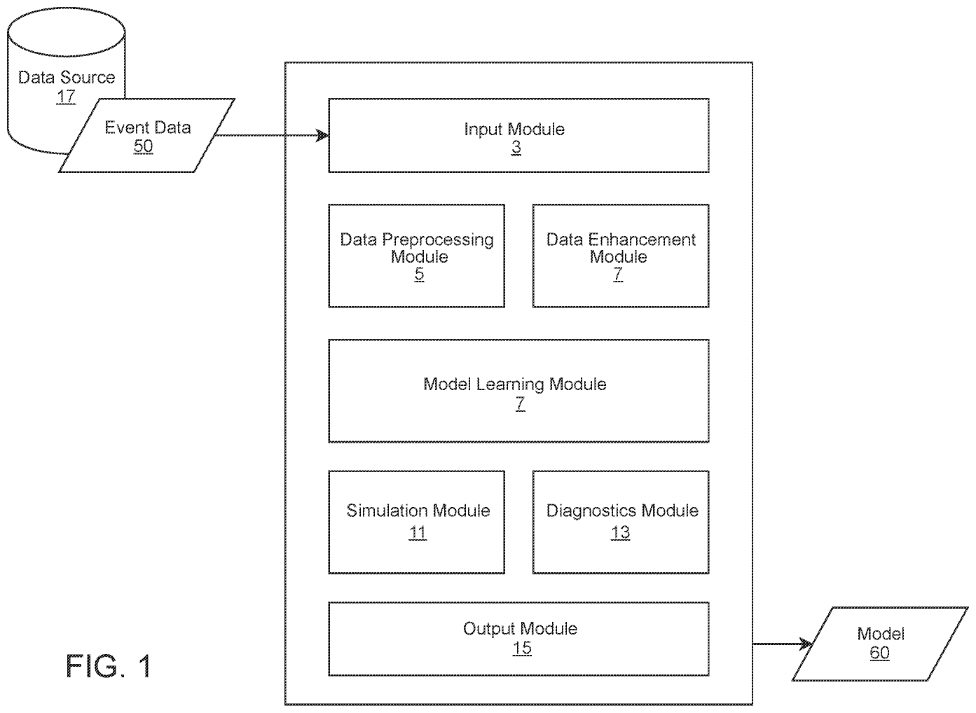

With reference now to FIG. 1, an exemplary system 1 for automatically modeling a process is shown according to an embodiment. In the illustrated embodiment, the system 1 comprises an input module 3, data preprocessing module 5, data enhancement module 7, model learning module 9, simulation module 11, diagnostics module 13, and an output module 15. As can be appreciated, these modules can comprise software modules implemented on programmable devices, such as one or more physical or virtual computers comprising a processor and memory. In some embodiments, one or more non-transitory computer readable media can comprise instructions that cause a processor to carry out steps which implement the modules. It is appreciated, however, that other configurations are possible. Broadly described, the system 1 is configured to receive event data 50 occurring as part of the process, and automatically generate therefrom a model 60 that describes the process and that can be used to perform descriptive, comparative, predictive and/or prescriptive analytics.

The system 1 is configured to receive event data 50 via input module 3. As can be appreciated, event data 50 can be stored on a data source 17, such as an external database. Accordingly, input module 3 can be configured to interface with data source 17 in any suitable manner to receive event data therefrom 50. The received event data 50 can subsequently be processed via data preprocessing module 5, data enhancement module 7, and model learning module 9 to produce a candidate model therefrom which describes various characteristics of the process. The operations carried out by these modules to produce the candidate model are described in more detail herein below.

The system 1 further includes a simulation module 11 that is configured to carry out simulations using the candidate module to predict the behaviour of the modeled process subject to different inputs. Moreover, a diagnostics module 13 is provided to allow calculating performance measures to allow validating performance of the candidate model. A validated model can subsequently be output via output module as a model 60 that can be used for subsequent comparative and prescriptive analytics. As can be appreciated, the output module 15 can be configured to output module 15 in different forms. For example, in some embodiments, the output module 15 can comprise an interface allowing the model 60 to be provided to other systems for further analysis. In some embodiment, the output module 15 can comprise a user interface, for example to output the model 60 and/or performance measures calculated therefrom for viewing by a user on a display device.

In more detail now, the event data 50 that is processed by system 1 can, for example, be generated by computing systems that gather transactional data on objects, events, and activity that is processed by a service system implementing the service process. In an embodiment, the event data 50 can correspond to granular transactional data that can be found in event logs. As can be appreciated, event logs can be stored in computing systems using different storage mechanisms. For example, event logs can be stored in a database that can comprise one or more related tables. In such tables, each line or row can correspond to an event, while each column corresponds to an attribute that has a value that describes characteristics of the event. Such characteristics can, for example, be used as features to train models such as machine learning models, as will be described in more detail hereinbelow. Accordingly, in the following disclosure, the term “row” will be understood to refer to an individual event recorded in event data 50, and the term “column” will be understood to refer to an attribute describing a characteristic of an event that can correspond to a feature for training a model such as a machine learning model.

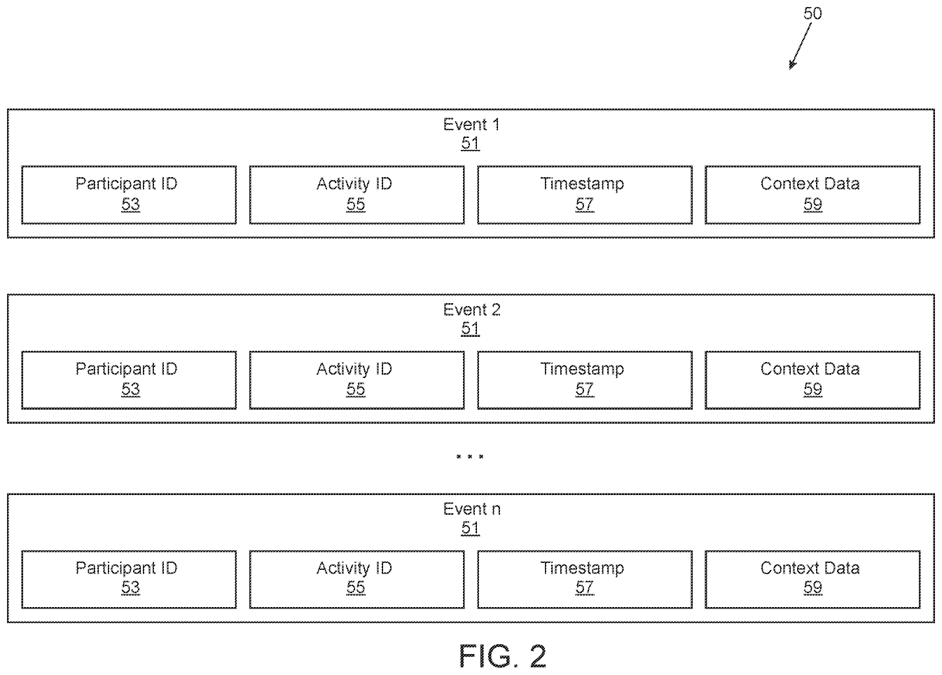

With reference to FIG. 2, exemplary event data 50 that can be received by system 1 is shown according to an embodiment. As can be appreciated, event data 50 can allow inferring the structure and dynamics of the process from which such event data was collected. To allow making such inferences, the event data 50 can comprise rows for a plurality of recorded events 51 and, for each row, columns comprising at least a participant 53 identifier, an activity identifier 55 and a timestamp 57. In some embodiments, the event data 50 can further include context data 59.

The participant identifier 53 uniquely identifies a participant that is involved in the event 51. Such a participant can correspond to a resource that is providing a service and/or to a customer that is receiving the service. As can be appreciated, any suitable identifier can be used to uniquely identify a participant. In some embodiments, the participant identifier 53 can comprise a number attributed to a given participant that is different from numbers attributed to other participants. As an example, if the service process corresponds to operating an emergency department in a hospital, the participant identifier 53 can comprise a case identifier, which is a unique number attributed to a patient during a specific visit to the emergency department. As another example, the participant identifier 53 can comprise a resource identifier, for example identifying a doctor or a nurse providing a service to the patient as part of a given event. As can be appreciated, a plurality of participant identifiers can be provided if there are a plurality of participants involved in an event. For example, where an event corresponds to a customer receiving a service from a resource, a first participant identifier can be provided for identifying the customer, and a second participant identifier can be provided for identifying the resource.

The activity identifier 55 uniquely identifies an activity concerned by the event 51. Such an activity can, for example, be associated with an individual service that is provided to a customer as part of the service process, or a station that the customer visits to receive such service. As an example, if the service process corresponds to operating an emergency department in a hospital, the activity can be an administrative or a medical service provided to a patient, for instance registration, admission, vital signs measurement. Depending on the nature of a service, one or more activities can be associated with such service. For example, an activity can correspond to a service as a whole having been provided to a customer, such as a patient having proceeded with registration in the emergency department. As another example, a first activity can correspond to a start of a service being provided to a customer, such as the emergency department admissions process starting for the patient, and a second activity can correspond to an end of the service being provided to the customer, such as the admissions process ending for the patient. As can be appreciated, any suitable identifier can be used to uniquely identify an activity. For example, the activity identifier 55 can comprise a number attributed to a given activity that is different from numbers attributed to other activities. As another example, the activity identifier 55 can comprise a label that is attributed to a given activity, such as a description of the name of the activity, that is different from labels attributed to other activities.

The timestamp 57 can provide an indication of the date and/or time at which the event occurs. As can be appreciated, the timestamp 57 can be provided in any suitable format. For example, the timestamp 57 can comprise a string of characters that conforms to any date and/or time format defined in the ISO 8601 standard, or can comprise a natural number that represents the Unix time, which is the number of seconds that have elapsed since 1 Jan. 1970 at midnight UTC.

The context data 59 can comprise additional columns that reflect characteristics of the participants (i.e. customers and/or resources) and/or of the activities (i.e. services provided to customers by the resources) concerned by an event 51. For example, if the service process corresponds to operating an emergency department in a hospital, the context data 59 can comprise columns including characteristics of the patient participating in an event, such as the patient's age, gender, address, etc. The context data 59 can further comprise columns including characteristics of a resource, such as information about the doctor or nurse providing service to the patient. As can be appreciated, columns of context data can correspond to static parameters in that their value does not change throughout the process. Columns of context data can alternatively correspond to dynamic parameters in that their values are subject to change during the process. An example of static parameters is a patient's age or gender which will remain the same throughout a visit to the emergency room. An example of a dynamic parameter is an assigned triage score, which may change as the patient is subjected to different activities in the emergency room. As will be described in more details below, context data 59 can be enhanced by the addition of system parameters derived from the event data 50. System parameters can correspond to parameters that the system itself is generating, for example describing the system's congestion.

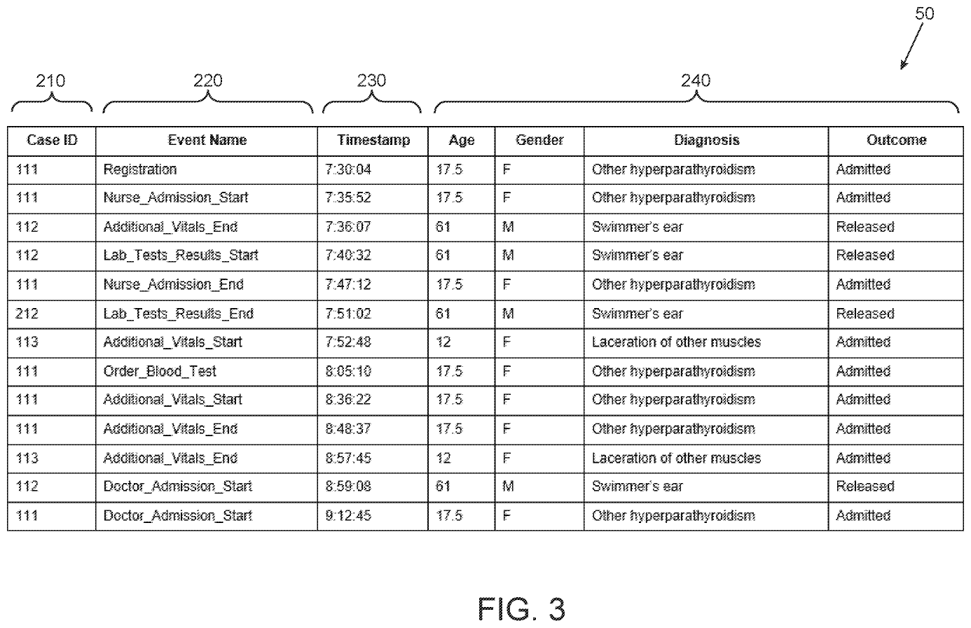

With reference to FIG. 3, an exemplary event log of an emergency department in a hospital is shown according to an embodiment. In the illustrated embodiment, the event log comprises event data 50 stored in a database and structured as a table. Each line or row of the table corresponds to an event 51, and seven columns are provided, each corresponding to an attribute that describes characteristics of the event. The first column 210 corresponds to a participant identifier 53. In the present embodiment, the participant identifier 53 is a case ID that comprises a number uniquely identifying a patient during a specific visit at the emergency department. The second column 220 corresponds to an activity identifier 55. In the present embodiment, the activity identifier is an event name that comprises a unique label describing a service in the process. The third column 230 corresponds to a timestamp 57. In the present embodiment, the timestamp 57 is a string of characters representing the time at which the event occurs in the format HH:MM:SS. As can be appreciated, columns 210, 220, 230, respectively corresponding to participant identifier 53, activity identifier 55 and timestamp 57, can comprise a minimum amount of data to describe the path of a patient through an emergency room process. For example, it can be seen that the patient corresponding to the case identifier 111 was provided the service named “Registration” at 7:30:04, was provided the service named “Nurse_Admission_Start” at 7:35:52, was provided the service named “Nurse_Admission_End” at 7:47:12, etc. A similar sequence of provided services can be followed for other patients associated with other case identifiers. Although not illustrated, it is appreciated that a resource identifier can also be associated with each activity.

The illustrated event log further includes additional columns 240 correspond to context data 59. In the present embodiment, the context data 59 comprises static parameters describing characteristics of patients, such as the age and gender of the patients. The context data 59 further comprises parameters describing characteristics of the services provided to the patient, such as the diagnosis attributed to the patient, and the outcome of the patient's visit. As can be appreciated, although such context data may not be required to describe the path of patients through the emergency room process, it can be used to predict various performance indicators, including length-of-stay of the patients, system utilization, etc.

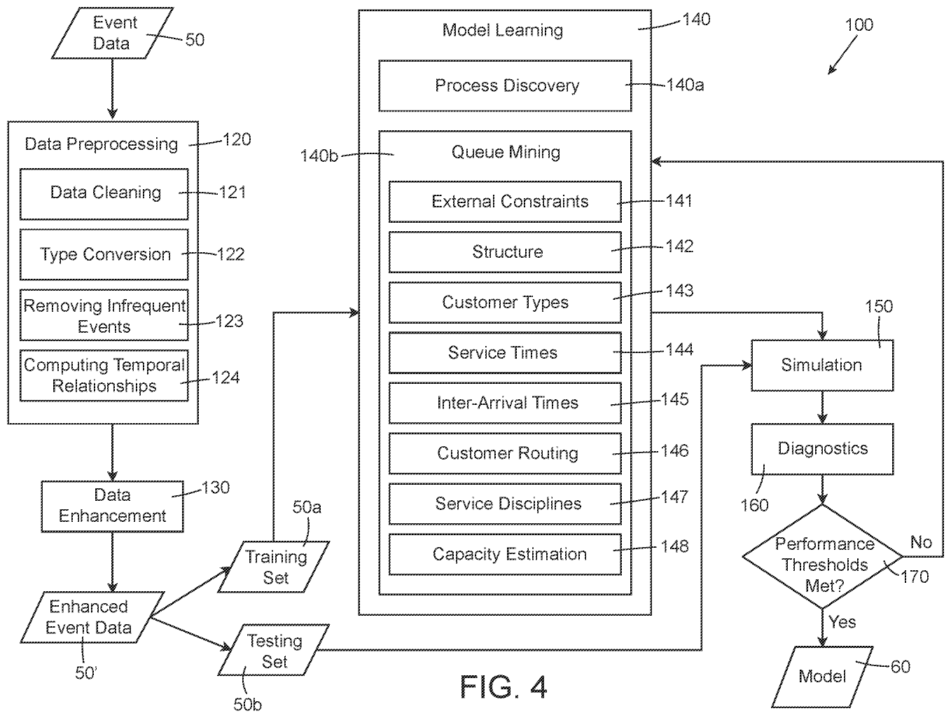

With reference now to FIG. 4, an exemplary method 100 for automatically generating a model of a process is shown according to an embodiment. As can be appreciated, the method 100 can be carried out via various modules of the above-described system 1. The method 100 can comprise a first step 110 of receiving, via input module 3, event data 50 describing events processed by a system implementing the process. Such data can be referred to as “historical” event data in that it corresponds to a record of actual events processed in the past by the system, and can be distinguished from “simulated” event data that merely seeks to emulate how the system would behave.

As can be appreciated, depending on the storage scheme used, event data 50 can be received in different forms. For example, event data 50 can be received in the form of an event log corresponding to a table in a database. In such an embodiment, receiving the event data can comprise connecting to the database and/or importing a table from the database corresponding to the event log. The columns of the table can be manually annotated by a user to indicate which columns correspond to the participant identifier, the activity identifier, and the timestamp. The columns can further be manually annotated to indicate additional context information. In some embodiments, the event data can be distributed among a plurality of tables, and receiving the event data can comprise combining or flattening data from the plurality of tables into a single table.

A second step 120 of the method 100 can comprise preprocessing the event data 50 via data preprocessing module 5. As can be appreciated, any suitable preprocessing steps can be applied. In the present embodiment, the data preprocessing 120 comprises four sub-steps (which can be implemented via sub-modules of preprocessing module 5), namely data cleaning 121, type conversion 122, removing infrequent events 123, and computing temporal relationships 124, although it is appreciated that other preprocessing steps are possible in other embodiments.

Data cleaning 121 can comprise completing or removing incomplete data. An event recorded in the event data 50 can have one or more missing values if does not specify a value in one of its columns, such as a participant identifier, an activity identifier and/or a timestamp. In some embodiments, events with a missing value can be removed from the event data 50. In other embodiments, a user can be provided with a request to fill in the missing value, or ignore the missing value if it isn't critical. For columns that represent values with a known type or domain, a value that is not of the required type or in the appropriate domain can be treated in the same way as a missing datum. In some embodiments, an outlier value can be treated in the same ways as a missing value. As can be appreciated, well-known anomaly detection methods can be applied to the values to identifier outlier values. For example, a probabilistic model can be created from the values, wherein a value that has a probability according to the model below a given threshold is considered an outlier value.

Type conversion 122 can comprise encoding certain values into a format that can be exploited by a machine learning module. For instance, if a column corresponds to a categorial attribute, such as a text label, the values of such attribute can be encoded before being submitted to machine learning modules. In some embodiments, if a categorial attribute can take a number of values, dummy attributes can be created corresponding to the number of values minus one. The categorial attribute can then be removed from the event data 50 replaced by the dummy attributes. Other approaches are also possible. For example, if a categorial attribute can take a number of values, one-hot attributes can be created for each of the possible values.

Removing infrequent events 123 can comprise removing, from the event data 50, rows corresponding to activities and/or sequences of activities that occur relatively infrequently. For example, a threshold can be provided or computed corresponding to a number of occurrences of activities or sequences of activities that is considered rare. The events corresponding to the activities and the sequences of activities that occur a number of times that is below the threshold can be removed from the event data 50. In some embodiments, the threshold can be computed by attempting to generate a model of the process with different thresholds and selecting the threshold that creates the model with the best performance as defined hereafter.

Computing temporal relationships 124 can comprise computing relationships between different activities in the process. For example, pairs of activities that directly follow one another in the event log can be identified and the frequency thereof can be computed.

A second step of the method 100 can comprise enhancing data 130 via data enhancement module 7. During this step, new attributes can be created from the event data 50, such as system parameters that correspond to parameters that the system itself is generating. As an example, a system parameter can include a parameter that describes system occupancy. The system occupancy can correspond to a measure of how many participants are currently in the system and can, for example provide indications of congestion, trend and seasonality in the process. The new, derived attributes corresponding to system occupancy information or measures can then be added to the event data 50 as new columns, including for instance a column indicating the occupancy of an activity at the time of each event, to produce enhanced event data 50′. In this fashion, the model of the process created from the enhanced event data 50′ can be aware of the new attributes/features relating to system occupancy. As an example, correlations between sojourn times (total length of stay durations) of patients that arrive during overlapping periods in time (e.g., the same hour) can be computed. This correlation can be added as a feature that will make the model aware of state and system congestion. As another example, if patients are not-pre-assigned to groups, patients can be grouped into clusters by their similarities in length-of-stay. Subsequently, the number of patients in each cluster over time can be added to the data as part of an additional enhancement step. In some embodiments, the new attributes describing system occupancy in the process can be computed according to the methods described in Senderovich, A., Beck, J. C., Gal, A., & Weidlich, M. (2019), “Congestion graphs for automated time predictions”, Proceedings of the AAAI Conference on Artificial Intelligence, 33(01), 4854-4861 (hereinafter “Senderovich et al., 2019”), the entirety of which is incorporated herein by reference. In particular, using modified queueing network mining techniques, a congestion graph can be created to represent a flow of resources in terms of events and labelled with performance information that is extracted from the event data 50. The extraction of the performance information can be grounded in a state representation of an underlying queueing system. The labels can then be transformed into attributes that can be added to the event data 50 to produce the enhanced event data 50′. Using such techniques, system occupancy-related attributes such as the number of patients in the system, their elapsed time, and their inter-arrival time can be added as features to help define the model of the process.

A subsequent step in the method 100 can comprise model learning 140 in which a simulation model of the process is generated from enhanced event data 50′ via model learning module 9. In the present embodiment, the enhanced event data 50′ is separated into two distinct sets of data: a training set 50a and a testing set 50b. As can be appreciated, the training set 50a can be used during the model learning step 140 to build the model of the process, whereas the testing set 50b can later be used to evaluate and/or validate the model, for example as part of the diagnostics step 160 that will be described in more detail hereinbelow. Separating the enhanced event data 50′ can be carried out in any suitable manner. For example, a predefined proportion of the resources can be selected randomly such that the events associated with the selected resources are placed in the training set 50a, and the remaining events are placed in the testing set 50b. In some embodiments, k-fold cross-validation can be used. For example, in 10-fold validation, ten training sets 50a and ten testing sets 50b can be prepared, in such a way that each one of the training sets 50a contains 90% of the events and each one of the testing sets 50b contains the remaining 10% of the events, and that none of the ten testing sets 50b contain shared events. As another example, the event data 50′ can be separated according to defined time periods. For example, the event data can be separated such that events occurring prior to a defined cut-off time (e.g., 3 months prior to the present date) are included in the training set 50a, and all remaining events after the defined cut-off (e.g., events that occurred between the present date and 3 months ago) are included in the testing set 50b.

In the present embodiment, model learning 140 is carried out in two sub-steps: a process discovery sub-step 140a and a queue mining sub-step 140b. The process discovery 140a sub-step can be carried out on a corresponding sub-module of model learning module 9, and generally involves estimating pathways and routing probabilities of activities from the training data 50a to produce a pre-model. The queue mining sub-step 140b can be carried out on a corresponding sub-module of model learning module 9, and generally involves enhancing or enriching the pre-model by fitting a plurality of queueing building blocks, as will be described in more detail hereinbelow.

As part of the process discovery sub-step 140a, a list of activities can be extracted from the appropriate column of the training data 50a. In some embodiments, an additional activity can be created to correspond with the beginning of the process, which is used by all customers and precedes all the other activities in sequences, and an additional activity can be created to correspond with the end of the process, which is used by all customers and follows all the other services in sequences. A pre-model can subsequently be estimated from the training data 50a to describe all possible pathways between the extracted activities and their corresponding routing probabilities.

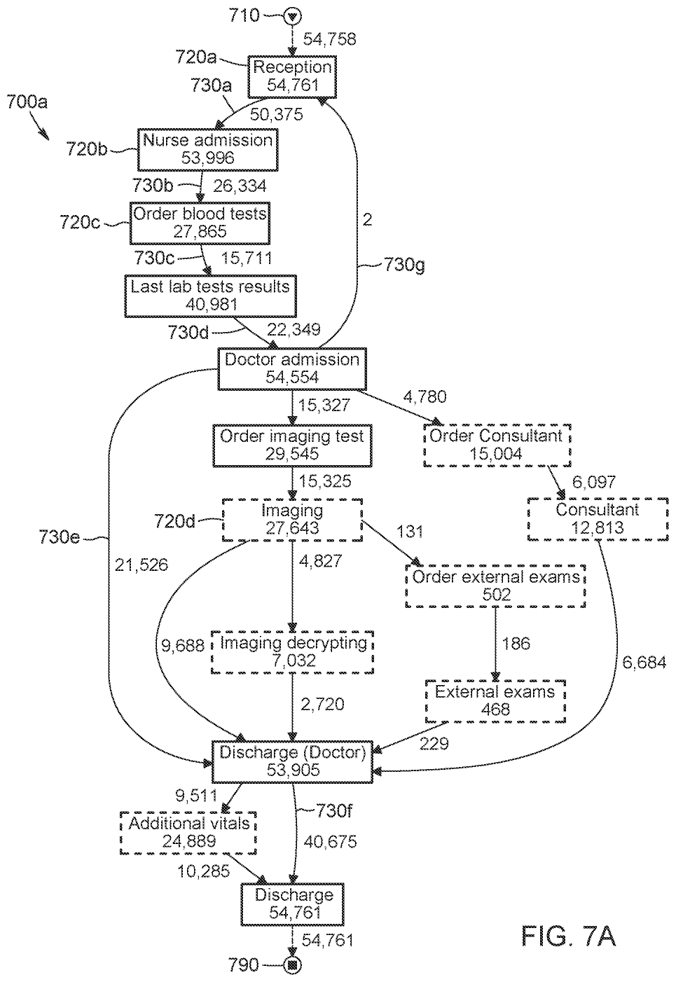

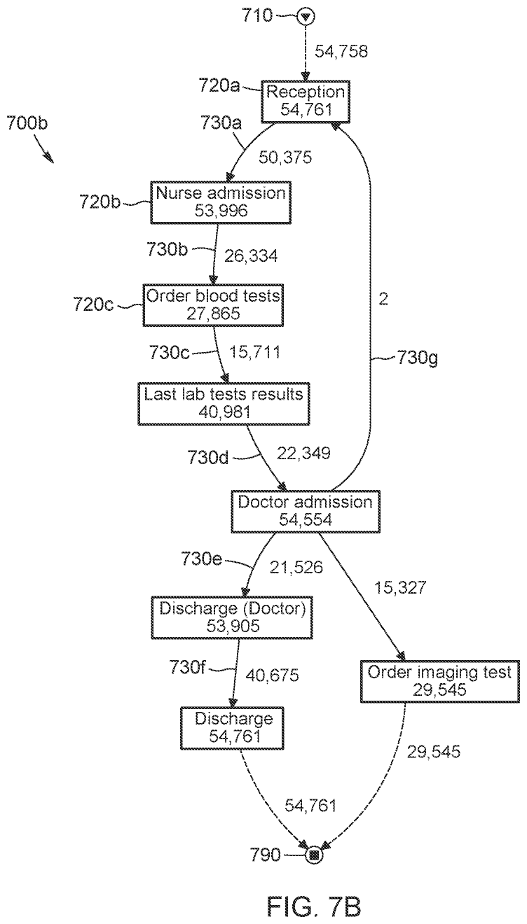

As can be appreciated, the pre-model can be constructed using any suitable format. For example, in some embodiments, a directed graph can be created. With reference to FIGS. 7A and 7B, exemplary pre-models 700a and 700b are shown according to a simple emergency room embodiment where the pre-models 700a and 700b are directed graphs. Each directed graph can include one vertex for each one of the activities extracted from the training data 50a, which can be labelled for instance with an activity name or identifier, e.g. 720a-d, and a plurality of arcs connecting the vertexes according to the different routing possibilities, e.g. 730a-g. Each arc represents an atomic pathway from a source vertex corresponding to a first activity to a target vertex corresponding to a second activity and indicate that a sequence in which the second activity immediately follows the first activity exists for at least one customer in the training data. For instance, arc 730a indicates that at least one customer appearing in the training data went directly from the “Reception” activity to the “Nurse admission” activity. Different process mining techniques can be used to discover the arcs. Each directed graph can additionally include one or more vertexes 710 representing arrival points and one or more vertexes 790 representing departure points.

Pathways as presented above can be identified in the pre-models 700a and 700b. For instance, arcs 730a, 730b, 730c, 730d, 730e and 730f together represent a frequent pathway of customers through the system. It can be appreciated that pre-models 700a and 700b can comprise cycles, e.g. the pathway corresponding to arcs 730a, 730b, 730c, 730d and 700g. Therefore, in embodiments using directed cyclic graphs as pre-models, pathways are defined to include all relevant graph-theoretical finite, open or closed directed walks, and not merely paths.

In the directed graphs corresponding to pre-models 700a and 700b, vertexes corresponding to activities can be labelled and/or decorated with respect to the frequency of the corresponding activities in the training data 50a. For instance, the activities corresponding to vertexes 720a, 720c and 700d occur respectively 54,761, 27,865 and 27,643 times in the training data, therefore the vertexes can be labelled respectively with “54,761”, “27,865” and 27,643. Assuming a frequency threshold of 27,750, vertexes 720a and 720c can additionally or alternatively be decorated, e.g. with a background and/or border colour or texture, to indicate that they correspond to frequent activities, whereas vertex 720d can additionally or alternatively be decorated, e.g. with a background and/or border colour or texture, to indicate that it corresponds to an infrequent activity. Arcs corresponding to routing possibilities can be labelled and/or decorated as well with respect to the frequency of the corresponding route from the source vertex to the target vertex. For instance, the route corresponding to arc 730a occurs 50,375 times in the training data, therefore the arc can be labelled with “50,375” and/or rendered with a proportional thickness. It can be appreciated that relative frequencies or probabilities can be used alternatively or additionally to absolute number of occurrences in labelling and decorating vertexes and arcs.

In some embodiments, the arcs can be discovered by applying the techniques described in Senderovich, A., Weidlich, M., Yedidsion, L., Gal, A., Mandelbaum, A., Kadish, S., & Bunnell, C. A. (2016), “Conformance checking and performance improvement in scheduled processes: A queueing-network perspective”, Information Systems, 62, 185-206 (hereinafter “Senderovich et al., 2016”), the entirety of which is incorporated herein by reference. Using such techniques, a partial ordering of the activities can be inferred from the sequence of activities that are observed in the training set 50a. A qualitative constraint network using an interval algebra, such as Allen's interval algebra, can be instantiated from the partial ordering, where each one of the services corresponds to one interval. This can allow discovering a total ordering on the lower and upper bound of the intervals, for example by computing the closure of the qualitative constraint network under relation composition. The arcs can be created from the total ordering. In some embodiments, where an activity is followed by a plurality of activities that may be used simultaneously, an additional vertex corresponding to a fork can be inserted in the directed graph, and where a plurality of services is followed by one activity that can start only once the plurality of activities has completed, an additional vertex corresponding to a join can be inserted in the directed graph. In some embodiments, where an activity is followed by a plurality of activities that may not be used simultaneously, the probability that each one of the plurality of activities follows the activity is computed and added to the directed graph, for example as an arc weight. In some embodiments, the directed graph with weighted arcs can be a probabilistic fork/join network.

In some embodiments, the pre-model can be further processed in order to filter out rare activities, paths and/or transitions. For example, the pre-model can be analyzed in order to identify vertexes that are visited infrequently in the training data, for example below a predetermined threshold (such as an absolute threshold or a threshold relative to other vertexes), and such vertexes can be removed from the pre-model. As an example, pre-model 700b correspond to pre-model 700a after filtering with a vertex-wise absolute threshold of 27,750, i.e. all vertexes corresponding to activities occurring less than 27,750 times such as 720d are removed. Similarly, the pre-model can be analyzed in order to identify arcs or transitions between vertexes that are followed infrequently in the training data, for example below a predetermined threshold (such as an absolute threshold or a threshold relative to other arcs or transitions, for example following calculation of a statistical parameter), and such arcs or transitions can be removed from the pre-model. If the directed graph of the pre-corresponds to a Markov chain, the arc or transition can be removed by setting its probability to 0, for example.

With reference once more to FIG. 4, once the pre-model has been created, the queue mining sub-step 140b can comprise applying queue mining techniques to fit a plurality of building blocks from the training data 50a to enhance the pre-model. The building blocks can be fitted, for example, using the techniques described in Senderovich et al., 2016. In the present embodiment, a total of eight possible building blocks 141-148 can be fitted, but it is appreciated that different building blocks and/or combinations thereof can be fitted in other embodiments. The eight building blocks 141-148 will be described in more detail hereinafter for exemplary purposes. For simplicity, they will be referred to as first, second, third, etc. building blocks, but it is appreciated that they are not being presented in any particular order. As can be appreciated, each building block can correspond to a submodule of the model learning module 9, and can be applied independently from one another.

In the present embodiment, a first building block 141 can relate to external constraints or delays. As can be appreciated, depending on the nature of the process, factors that are external to the process may introduce delays in the provision of services that are reflected in the training data 50a. As an example, if the process is a service process corresponding to operating an emergency department in a hospital, such external factors can correspond to external consults, bed blocking, and ambulance diversion, among other. As can be appreciated, consultations by professionals outside of the emergency department are external to the patient flow process in the emergency department yet can introduce delays therein. When a destination ward outside the emergency department is full, departure of patients from the emergency department can be delayed. Similarly, when external decisions are made to temporarily divert ambulances, the patient arrival process can be affected. Given that such constraints are exogenous to the emergency department service process, the pre-model can be adjusted such that the model of the process reflects the effect of such external constraints.

As can be appreciated, different steps can be carried out to fit the first building block 141 and adjust the pre-model to account for impacts of external constraints or delays. In an embodiment, a first step can comprise discovering external constraints that can have an effect on the training data 50a, for example based upon user input or additional information (knowledge or data). A second step can comprise adjusting the model to capture the effects of such constraints on the process. One example in the context of emergency departments is specialist physicians. In the first step, it can be observed that emergency patients seen by a specialist experience higher waiting time. As specialists are not under direct supervision of the management of the emergency department, their service is an exogenous constraint on the system. In the second step, the specialist service can be added to the visit characteristics, for example as one or more additional context data attributes in the training data 50a. These additional attributes can later be used as features feeding models such as machine learning models that describe customer processing times and routing.

A second building block 142 can relate to the structure of the queueing network describing the process. As can be appreciated, fitting this building block can allow discovering the queueing network, including relevant stations therein. In a first step, historical visits of customers to stations can be identified. In a second step, unique stations that are frequently visited, i.e. above a predetermined frequency threshold, can be integrated into a network of queues defining the structure of the queueing network. Similarly, unique stations that are infrequently visited, i.e. below the predetermined frequency, can be omitted (i.e. removed) from the queueing network structure. Fitting this building block can further comprise discovering relevant pathways and/or transitions in the queueing network. Pathways and/or transitions between stations can be identified from historical visits, and pathways and/or transitions that are frequently followed, i.e. above a predetermined frequency threshold, can be integrated into the network of queues. Similarly, pathways and/or transitions that are infrequently followed, i.e. below the predetermined frequency, can be omitted (i.e. removed) from the queueing network structure, for example by setting their probability to 0.

A third building block 143 can relate to customer types. As can be appreciated, some jobs and/or customers that differ in certain characteristics may be treated the same way by the process. For example, patients that are characterized as male or female may be processed identically in a service process. Similarly, segments of customers can exist that do not differ in known characteristics, but may still be treated differently by the process. For example, patients may be prioritized at a certain point in the service process if they previously received a service over other patients that did not previously receive such service. Accordingly, given that processes can offer different service classes (e.g. priority, routing and service times) to different customer types, the pre-model can be adjusted to model such customer types. Similarly, given that processes can be the same for different customer types, the pre-model can be adjusted to group pathways for such customers. As can be appreciated, the actual priority rules used by the process may differ substantially from the ones stated in organizational policies or expected by organizational management. Moreover, such prioritization may depend on other factors such as the occupancy level of the system, the available process resources at a certain time, etc. Thus, discovering the priority structures that are actually used in the process can be useful for accurately modeling the system.

As can be appreciated, different steps can be carried out to fit the third building block 143 and account for different possible service regimens applied to different customer types. In some embodiments, machine learning can be applied to discover the different customer types from the training data 50a. This can be a two-step process. In a first step, machine learning classification and clustering algorithms can be used to identify customer types that are treated differently in one or more parts (for example at one or more different stations) of the process. In some embodiments, customer groups can be identified from existing characterization (such as from context data 59) and/or from observed process data, such as a path of the customer through the process. In a second step, once the different customer types have been identified, such customers can be grouped and the pre-model can be adjusted accordingly. In order to adjust the pre-model, machine learning algorithms can be integrated therein, such as random forests that can predict and generate service times based on generalizations that use the derived customer types. The service time, routing, and inter-arrival time probability distribution functions can then be conditioned on the newly discovered customer types.

A fourth building block 144 can relate to service times. Broadly described, the service time distribution for each service and for each customer type can be estimated and applied to the pre-model. In some embodiments, statistical or machine learning methods can be applied to the training data-set 50a, the pre-model and/or the customer types, to predict the duration of a given service for a given customer type. For example, neural network can be used to sample service times for arriving customers. Additionally or alternatively, a regression task can be performed for each customer type identified by building block 143. For instance, in embodiments where building block 143 creates clusters of customers, a kernel density estimator can be fitted to each created cluster to predict the value of the service time for customers in the given cluster for each service. In some embodiments, a plurality of different methods can be used to estimate the service times and generate a plurality of models. The models can subsequently be tested using out-of-sample data (i.e. using testing data set 50b), and the model that fits the out-of-sample data best can be retained.

A fifth building block 145 can relate to inter-arrival times. Broadly described, inter-arrival time distribution can be estimated for each service and for each customer type. In some embodiments, machine learning methods, for example adversarial neural networks such as TimeGAN or long short-term memory units in Recurrent Neural Networks can be applied to the training set 50a to predict arrival time and volume of customers at different services in the process. This building block can be broken into three steps. In a first step, the arrival process can be modeled using customer, time, and system characteristics (including the derived customer type from the previous building block). A second step can comprise training a predictive model, such as a Recurrent Neural Network that uses these characteristics. In a third step, the predictive model can be used to generate customer arrival times, and prediction intervals can be provided.

A sixth building block 146 can relate to customer routing. As can be appreciated, the routing of customers through the process may change when the system is busy or congested. Accordingly, the pre-model can be adjusted to allow modeling state-dependent routing. In an embodiment, routing from each station in the process to each other station in the process can be identified for each customer type. As can be appreciated, different methods can be applied to identify customer routing. In some embodiments, machine learning algorithms, for instance k-nearest neighbours classification, can be applied to the training set 50a and the customer types to discover historical paths taken through the process by different classes of customers under different circumstances, for example depending on how busy the process is at a given time. In some embodiments, a statistical approach, e.g. a frequentist approach, can additionally or alternatively be applied to estimate Markovian routing probabilities. In an embodiment, modeling customer routing can comprise three steps. In a first step, historical routing schemes can be inferred using standard process mining techniques. A second step can comprise enhancing the inferred routing schemes with additional system and customer characteristics (e.g., derived customer type) to better capture customer routing in the model. A third step can comprise generalizing the routes observed in the data (and enhanced in the second step) using machine learning methods such as k-nearest neighbours and random forests and/or a statistical approach. This can allow creating appropriate routing rules for the simulation model.

A seventh building block 147 can relate to service discipline. As can be appreciated, each station in the process can apply different service or queueing disciplines (such as priority, first come first served, last come first served, shortest job first, round robin, etc.) when providing services to a customer. Accordingly, the pre-model can be adjusted to account for different service disciplines that are used at each station for different customer types. Queue mining algorithms can be applied to the training set 50a to discover the service discipline for services provided at each station in the system. In some embodiments, a general discipline function can be learned from the training set 50a for each station without specifically identifying the exact queueing discipline that is being used. This can allow learning any service policy, as long as its drivers are recorded in the training data 50a. In some embodiments, if a service at a station is offered by a plurality of servers, the service discipline can specify whether there is a unique queue for all resources waiting to use the service or whether there is one queue for each server. Service disciplines may depend on system and customer characteristics. By default, the pre-model can include a first-come first-served discipline applied to each station. Once a service discipline is identified for the stations, the pre-model can be adjusted by updating each station to use the corresponding identified service discipline.

An eighth building block 148 can be related to capacity. As can be appreciated, each station can have different potential and achievable processing capability that can vary at different times and for different customer types. For example, a station can have several resources, possibly of different types, with the composition changing during the day due to shifts. More specifically, in the context of operating an emergency department, the capacity of a service can be different depending on the time of day, accounting for personnel shifts, or depending on the time of year, accounting for personnel vacations. Accordingly, the pre-model can be adjusted to model time-varying capacity and aspects such as shifts and resource variation. As can be appreciated, different techniques can be applied to identify and/or infer capacity. In some embodiments, capacity level of each station at each point in time may be available from the context data. In other cases, techniques such as inverse optimization can be applied to the training set 50a to infer the capacity of each of the services as well as daily fluctuations and seasonal variations in the capacity. In some embodiments, inferences are performed to compensate missing data. For example, if no capacity is recorded in the data, state-dependent infinite server queueing models can be used to approximate the demand to capacity ratio. Such models can use estimates of service times to capture occupancy in the system and its impact on present and future customers.

The building blocks described above can be fitted according to different levels of complexity depending on the data that is available. As can be appreciated, each building block can have different data requirements, and individual building blocks can be fitted only if their data requirements are met. In other words, the sub-step 140b can comprise a preliminary step of determining attributes available in training dataset 50a, identifying one or more building block from among a plurality of buildings blocks whose data requirements are fulfilled by the available attributes, and fitting the one or more identified building blocks. In this fashion, when data is missing, the building blocks can degrade to an aggregated black-box model, i.e. they can correspond to basic techniques used in queue mining for single-station queues.

The model that results from the model learning step 140 will be referred to hereinafter as a candidate model and can, for example, comprise a queueing network with Markovian routing. In some embodiments, the queueing networks can be composed of the following station types: infinite-server queues, a processor sharing single resource queue, a finite-server first-come first-served (FCFS) queue. It is appreciated that other station types are possible in other embodiments, such as queues with priority discipline, reneging, etc. Such station can have context, state and time dependency embedded in its arrival and service processes. As described above, auxiliary attributes from the training dataset 50a can be used to build context and mine the state of the system, and time dependencies can be created by grouping by time of day and day of the week. Moreover, machine learning methodology (such as decision trees, random forests and K-means clusters) that uses system occupancy-related features that come from queueing theory, can be used to improve the ability to sample arrivals and service times.

As can be appreciated, a possible benefit of allowing state and time dependencies to be introduced into the model is the ability to circumvent situations when capacity data is limited or entirely missing. For example, using infinite server stations, the length of stay can be modeled in a system where the length of stay of each arriving customer depends on the number of customers of similar type in the system, and their current length of stay. This way, correlations that arise in congested systems can be captured indirectly despite the possible lack of explicit capacity data.

Once a candidate model is generated following the model learning step 140, the method 100 can comprise a subsequent step 150 of simulation which can include generating alternative behaviours of the system using the candidate model via simulation module 11. Since the queueing model defined therein is well-defined, standard approaches for queueing simulation can be applied. As can be appreciated, an advantage of the candidate model is that it can generate data using a combination of simulation and machine learning. For example, as explained above, length-of-stay per station can be modeled in the candidate model as a function of contextual features and state features. This aspect of the model can be based on an ensemble of regression trees, such as random forests. Thus, for every new arrival of a customer, the random forest model can predict the length-of-stay of the arriving customer based on various features (such as age, gender system state, etc.). In some embodiments, noise can be added to such predictions, for example from a noise bucket derived from historical data. This can allow generating new observations under the assumption that the noise is stationary, and the only elements of the model that depend on the context and state are the means of the length-of-stay distributions.

A final step 160 of the method 100 can comprise performing diagnostics on the candidate model via diagnostics module 13. This can comprise validating the candidate model using in-sample and out-of-sample performance measures, including histograms, confidence intervals, Q-Q plots, hypothesis testing (e.g. Kolmogorov-Smirnov test for comparing distributions), predictive accuracy measures such as mean squared error, etc. In an embodiment, candidate model can be diagnosed by applying the candidate model to data included in the testing set 50b to make predictions. The predictions of the candidate model can then be compared 170 to the actual historical event data 110. For example, performance metrics can be computed based on differences between the predictions and the actual event data 110. As can be appreciated, if the candidate model meets predetermined thresholds in the performance metrics, the model can be retained and output as a final model 60, for example via output module 15. In some embodiments, for example if one or more performance metrics are below the predetermined threshold, the method 100 can comprise returning to the model learning 140 step to further refine the candidate model. For example, once a model is created, the model can be simulated to generate multiple datasets. Performance indicators, such as the length of stay of customers in the system, can be calculated.

If the distribution of these performance indicators does not match the originating data, it can be concluded that the model is biased and the model can be re-fitted using a different set of features or by replacing one of the building blocks. One example of such replacement could be using a different machine learning algorithm that models service times or engineering additional features to be used in the algorithm. Cross-validation can be used to tune the hyperparameters of the various building blocks. The resulting model 60 can be accurate in both in- and out-of-sample, providing both descriptive and predictive power for analysis purposes.

As can be appreciated, the model 60 can be used to help make predictions about the effects of different interventions on the process. An intervention can correspond to a modification of the process or of the conditions under which the process is executed that can have the potential of impacting the performance of the process. Examples of interventions include speeding up or slowing down services, changing the routing between services for certain customer types, changing the capacity of services, changing the service discipline of services, or changing the resource types, among others. As a more specific example, if the process to be analyzed corresponds to the operating of an emergency department in a hospital, an administrator may want to know the effect of running the emergency department with a different number of doctors at a specific time of day, or the effect of changing routing and priority policies, or the effect of a greater proportion of patients necessitating a consult.

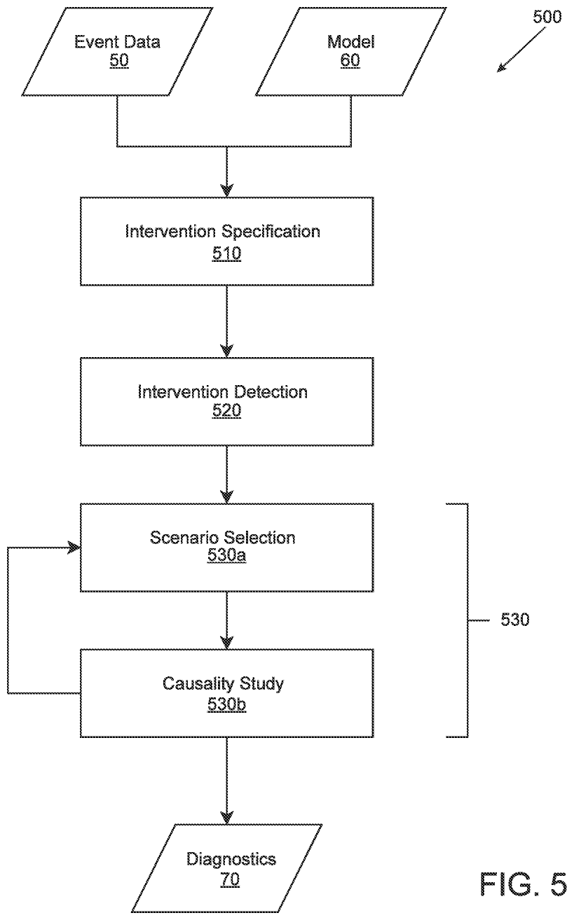

With reference to FIG. 5, an exemplary method 500 for predicting impacts of interventions on a process is shown according to an embodiment. As can be appreciated, the method 500 can be carried out using the model 60 and/or historical data 50 as an input. Using these inputs, the method can allow estimating impact of specified interventions based on historical data and/or can allow estimating impact based on designed experiments to evaluate “what-if” questions. In some embodiments, the method 500 can allow automatically proposing interventions, and the impact of such proposed interventions can be evaluated.

A first step 510 of the method 500 can comprise intervention specification in which the interventions to evaluate are defined. In some embodiments, the interventions can be user-specified. For example, a user can specify affected model blocks in a user-friendly intervention language, by specifying where one or more interventions could be applied (arrivals, resource types, and service times) and for which they would like to run a “what-if” analysis. In some embodiments, possible interventions can be identified from the building blocks 141 to 148 and can be suggested to the user, who can subsequently select from among the suggestions. In some embodiments, interventions can be proposed for external constraints. As can be appreciated, careful consideration can be given to interventions that are related to external constraints. Inducing external partners to change their processes in order to relax constraints on the studied system may require analysis that captures the impact of these external constraints on the studied system. Accordingly, a discussion with external partners can be useful only once this impact is quantifiable.

A second step 520 can comprise intervention detection, where ‘natural’ occurrences of user-specified intervention conditions can be detected in historical data. More specifically, the event data 110 can be analyzed to identify historical times periods where conditions corresponding to the user-specified intervention existed in the historical event data 110. For example, if an intervention corresponds to an increase in the arrival rate for a certain type of resources, there may have been historical periods where such increased arrival rates were observed in the event data 110. If a historical occurrence can be identified, statistical comparison of the system performance under these conditions and of the system under baseline (i.e. as-is) conditions can be performed, and these comparisons can be output as diagnostics 70. As can be appreciated, periods identified in this manner can be treated as “natural experiments” and can allow making predictions about the likely result of the intervention with a high level of certainty.

As can be appreciated, it is possible that no historical occurrences can be identified in event data 110 for a specified intervention and/or for a specified combination of interventions. In such cases, a subsequent step 530 can comprise a simulation-driven analysis in which the model 60 is used to infer the impact of different possible interventions such that diagnostics 70 can be produced therefrom. In this fashion, a simulation-driven analysis of the intervention impact can be provided as apposed to a data-driven analysis.

In an embodiment, the simulation-driven analysis 530 can comprise a first sub-step 530a of scenario selection. As part of this sub-step, one or more interventions (referred to as scenarios) can be selected for use as part of the simulation. In an embodiment, the scenarios can be user-specified. An intervention-aware simulation model can subsequently be constructed to allow simulating the joint impact of the selected interventions. Returning to the example of an emergency department, if a user only selected a staffing intervention for doctors, and evidence for having varying values for the number of doctors is found in the event data, this information can be integrated into the simulation model such that a what-if analysis can be executed. The user can observe a range of possible values with (or without) confidence intervals. This can allow illustrating the impact of changing the values of staffing on various performance measures.

A second sub-step 540b can comprise a causality study. In this subs-step, the simulation model can be used to project the system performance from the observed level of interventions to performance under different levels of interventions. In other words, a data-driven simulation model, such as the model 60, is used as a causal model to test the impact of different interventions. As can be appreciated, when the simulation executes, confidence intervals for the scenarios that were not observed in the event data 50 can be provided. The resulting diagnostics 70 can be observed and the simulation model can be changed again until the user can examine all interventions of interests, along with these respective impacts.

In some embodiments, it may not be possible to infer impact of certain interventions based on the existing data. In such cases, the method can comprise providing recommendations on the collection of missing data. The method can further comprise providing suggestions for short term changes that can help with the collection of additional relevant data. Such data can enable steps for data-based comparisons identified in the previous steps.

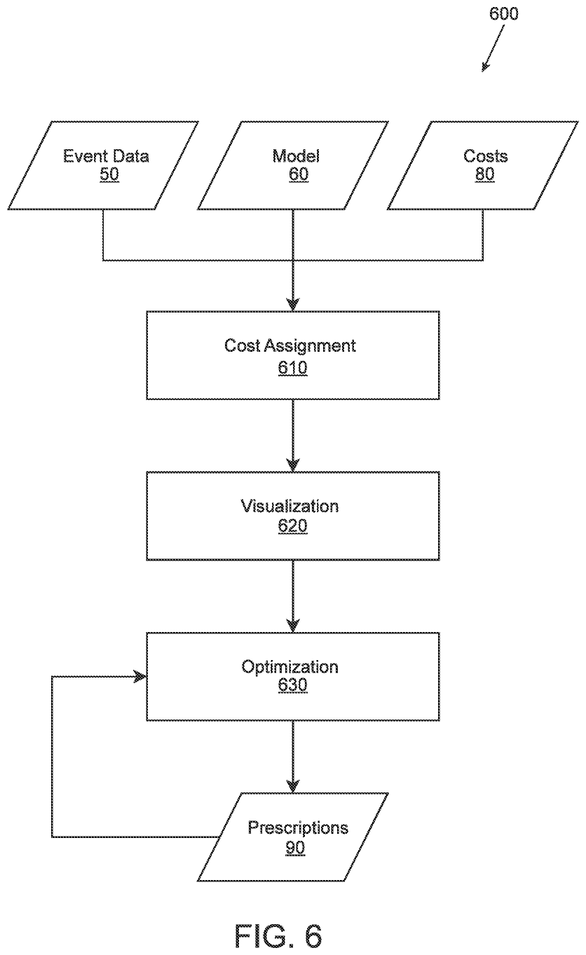

As can be appreciated, by producing diagnostics 70 relating to different possible interventions, trade-offs between costs and benefits can be evaluated in order to identify optimal levels of interventions within user-specified constraints, and given costs assigned to each of the intervention parameters. With reference to FIG. 6, an exemplary method 600 for optimizing intervention in a process is shown according to an embodiment. The method 600 can receive three inputs: event data 50; model 60, including feasible interventions identified according to method 500; and a set of use-specified costs 80.

A first step 610 of the method 600 can comprise cost assignment, where the user can input different costs 80 that are matched against the building blocks in the model 60. A second step 620 can comprise visualization, in which performance measures are used to display the benefit of different interventions, including confidence intervals. This can be done by visualizing trade-off curves of different interventions and their economic and statistical significance impact of performance measures. A final step 630 can comprise optimization in which interventions that would aim at optimizing with respect to the pre-defined costs and objectives can be found, and corresponding prescriptions 90 can be made.

The methods and systems described above can allow for automated data-driven modeling, analysis, and optimization of processes that are common in work systems such as service systems and often modeled as complex queueing networks. The described systems and methods can mine event logs generated by information systems to discover process structure and construct predictive models for their operational characteristics. Subsequently, two types of models (structural and predictive) can be used to simulate and forecast future behaviour of the process under a variety of conditions and interventions, as well as to identify optimal operational parameters.

In the described embodiments, the systems and methods apply techniques that combine study fields such as queueing theory, statistics, machine learning, and process mining. Specifically, the described embodiments utilize queue mining, which is a field that studies the combination of the above with an emphasis on the discovery of useful queueing networks from data, and on the application of these models to enhance models such as machine learning models for prediction and sampling.

Although particular examples have been provided (i.e. in the context of an emergency department), it will be appreciated that the described systems and methods can be applied to a wide variety of other work systems such as healthcare (long-term care homes), cloud computing (data services), and supply-chain management systems, among others. Given that a focus is on modeling processes in which queues are prevalent, applications are also possible for use in manufacturing.

Although particular advantages and applications of the invention have been explicitly described herein, other advantages and applications may become apparent to a person skilled in the art when reading the present disclosure. The invention is not limited to the embodiments and applications described, and one skilled in the art will understand that numerous modifications can be made without departing from the scope of the invention.

1. A method for training a simulation model of a process, comprising:
receiving historical event data corresponding to activities processed by a work system, said historical event data comprising a plurality of events and, for each event, values corresponding to a plurality of attributes, said plurality of attributes including at least a participant identifier, an activity identifier and a timestamp;
processing the historical event data to estimate system occupancy information and enhancing the historical event data by adding the estimated system occupancy information as at least one additional context attribute;
separating the enhanced historical event data into a training dataset and a testing dataset;
building the simulation model by extracting activities and estimating participant pathways and routing probabilities between the activities, from the training dataset; and
enhancing the simulation model by processing the training dataset to remove at least some of the participant pathways based on frequency, and to estimate durations of activities in the participant pathways.

2. The method according to claim 1, comprising preprocessing the historical event data prior to extracting system occupancy information, wherein preprocessing the historical event data comprises at least one of:
identifying in the historical event data at least one event with a missing value for at least one attribute and either prompting a user to input the missing value or removing the at least one event from the historical event data;
identifying in the historical event data at least some values provided in a first format, and encoding the at least some values into a second format that is different than the first format, said second format being adapted for being exploited via machine learning;
identifying in the historical event data a subset of the activities that occur with a frequency below a predefined threshold and removing from the historical event data events that correspond to the subset of the activities; and
identifying in the historical event data at least one pair of activities that directly follow one another for participants, and adding to the historical event data a temporal relationship between the first activity and the second activity.

3. The method according to claim 1, wherein the occupancy information comprises an indication of at least one of congestion, trend and seasonality in the process.

4. The method according to claim 1, wherein processing the historical event data comprises calculating correlations between sojourn times of participants in the work system during overlapping periods and adding the calculated correlation to the historical event data as an additional context attribute.

5. The method according to claim 1, wherein processing the historical event data comprises:
if the participants are not pre-assigned to groups, grouping participants in the work system by similarities in length-of-stay and adding a number of participants in each group over time to the historical event data as an additional context attribute.

6. The method according to claim 1, wherein enhancing the simulation model comprises using queue mining to fit a plurality of queueing building blocks from the training dataset.

7. The method according to claim 6, further comprising:
determining attributes available in training dataset,
identifying one or more queueing building block from among the plurality of queueing buildings blocks whose data requirements are fulfilled by the available attributes, and
fitting the one or more identified queueing building blocks.

8. The method according to claim 6, wherein the plurality of queueing building blocks comprises at least one building block configured to discover and group customer types through machine learning by applying a clustering algorithm.

9. The method according to claim 6, wherein the plurality of queueing building blocks comprises at least one building block configured to predict durations of activities for different customer types using a model configured to sample service times for participants arriving at activities in the work process, said model being trained on enhanced historical event data comprising context attributes including a service time distribution for each activity and for each customer type.

10. The method according to claim 9, wherein predicting the durations of activities for the different customer types comprises performing a regression task for each of the different customer types.

11. The method according to claim 6, wherein the plurality of queueing building blocks comprises at least one building block configured to use a model to predict arrival times of participants to different activities in the work process, said model being trained on enhanced historical data comprising context attributes including arrival time distributions for each activity and for each customer type.

12. The method according to claim 11, wherein predicting arrival times for different customer types comprises using a generative adversarial network algorithm.

13. The method according to claim 6, wherein the plurality of queueing building blocks comprises at least one building block configured to model routing rules for a given customer type by:
inferring historical routing schemes using process mining;
enriching the inferred routing scheme with the customer type; and
generalizing the routes using machine learning.

14. The method according to claim 6, wherein the plurality of queueing building blocks comprises at least one building block configured to model different service disciplines applied at different activities for different customer types.

15. The method according to claim 6, wherein the plurality of queueing building blocks comprises at least one building block configured to model external constraints that introduce delays in the activities in the process.

16. The method according to claim 6, wherein the plurality of queueing building blocks comprises at least one building block configured to discover a queueing network structure describing the work process by identifying activities in the historical event data that occur with a frequency above a predetermined threshold and integrating the identified activities in a network of queues.

17. The method according to claim 6, wherein the plurality of queueing building blocks comprises at least one building block configured to model time-varying capacity of different activities in the process.

18. The method according to claim 1, further comprising simulating the process by:
applying the testing dataset to the simulation model to generate predictions therefrom,
identifying differences in predictions made by the simulation model and actual historical event data,
calculating performance metrics to quantify said differences, and
refining the simulation model if the calculated performance metrics are below a predetermined threshold.

19. The method according to claim 1, further comprising evaluating an impact of process interventions by:
specifying interventions;
determining whether the historical event data contains events that correspond to the interventions;
responsive to determining that the historical event data contains events that correspond to the interventions, extracting an impact of the interventions from the historical event data;
responsive to determining that the historical event data does not contain events that correspond to the interventions, simulating the process with the interventions using the simulation model to predict the impact of the interventions; and
suggesting additional process data that needs to be collected to evaluate the impact of the interventions.

20. The method according to claim 19, further comprising deriving prescriptions by:
determining possible interventions;
specifying a cost function for the possible interventions;
specifying a performance function for the work process;
simulating the work process with at least one of the possible interventions using the simulation model to predict an impact of the at least one of the possible interventions; and
determining that a subset of the possible interventions optimizes a ratio of the performance function and the cost function.

21. A system for training a simulation model of a process, comprising:
an input module configured to receive historical event data corresponding activities processed by a work system, said historical event data comprising a plurality of event and, for each event, values corresponding to a plurality of attributes, said plurality of attributes including at least a participant identifier, an activity identifier and a timestamp;
a data enhancement module configured to process the historical event data to estimate system occupancy information and enhance the historical event data by adding the estimated system occupancy information as at least one additional context attribute; and
a model learning module configured to separate the enhanced historical event data into a training dataset and a testing dataset, the model learning module comprising a process discovery submodule configured to build the simulation model by extracting activities and estimating participant pathways and routing probabilities between the activities from the training dataset, and a queue mining submodule configured to enhance the simulation model by processing the training dataset remove at least some of the participant pathways based on frequency, and to estimate durations of activities in the participant pathways.

22. A non-transitory computer-readable medium having a simulation model trained according to the method of claim 1 and instructions stored thereon, said instructions, when executed by a processor of a computing system, causing the computing system to:
access interventions and historical event data used to train the simulation model;
determine whether the historical event data contains events that correspond to the interventions;
responsive to determining that the historical event data contains events that correspond to the interventions, extract an impact of the interventions from the historical event data; and
responsive to determining that the historical event data does not contain events that correspond to the interventions, simulate a process with the interventions using the simulation model to predict the impact of the interventions.
